(19) United States
(12) Patent Application Publication
(10) Pub. No.: US 2024/0368510 A1
(43) Pub. Date: Nov. 7, 2024

(54) MICROFLUIDIC DEVICES WITH PARTIALLY ENCLOSED MICROFLUIDIC CHANNELS AND METHODS FOR FORMING PERFUSABLE VASCULAR NETWORKS

(71) Applicant: QUREATOR, INC., San Diego, CA (US)

(72) Inventors: Kyusuk BAEK, San Diego, CA (US); Noo Li JEON, Seoul (KR); Byungjun LEE, Seoul (KR)

(21) Appl. No.: 18/566,607

(22) PCT Filed: Jun. 1, 2022
(86) PCT No.: PCT/US2022/031842

§ 371 Date: Dec. 1, 2023

Related U.S. Application Data
(63) Continuation-in-part of application No. PCT/US21/35277, filed on Jun. 1, 2021.

(51) Int. Cl. C12M 3/06; C12M 3/00

(52) U.S. Cl. CPC C12M 23/16; C12M 21/08

(57) ABSTRACT

A device includes a substrate having a top surface and a bottom surface opposite to the top surface; a first microfluidic channel and a second microfluidic channel defined on the substrate. The second microfluidic channel is distinct from the first microfluidic channel, and is in contact with, and substantially parallel to, the first microfluidic channel. A method for forming an endothelialized microchannel, a method for forming a vascularized spheroid, and a method for forming a vascularized organoid are also described.

### RELATED APPLICATIONS

This application is a continuation-in-part of PCT Application No. PCT/US2021/035277, filed Jun. 1, 2021, entitled “Microfluidic Devices with Partially Enclosed Microfluidic Channels and Methods for Forming Perfusable Vascular Networks,” which is incorporated by reference herein in its entirety.

### TECHNICAL FIELD

This application relates to microfluidic devices and methods for using the same. In particular, this application relates to methods for culturing cells or tissues using such microfluidic devices and methods for processing cells or tissues using such microfluidic devices, including co-culturing cells or tissue using a microfluidic device.

### BACKGROUND

Microfluidic devices having various structures of a microfluidic channel, chamber or reservoir have been used in various fields such as three-dimensional cell co-culture. By using a microfluidic device, cells constituting a specific tissue or organ are cultured in vitro for study and research of the functions, characteristics, and epidemiological and physiological cell responses of the tissue or organ, and also used to replace an animal test in new drug development.

However, conventional microfluidic devices for cell culture require a microfluidic channel that defines at least four sides of a volume in which cells are cultured. Thus, the spatial activity of cells is reduced or restricted. In addition, such a conventional microfluidic device requires a separate culture medium channel through which cell culture medium is provided. In conventional microfluidic devices, the cell culture medium may not be provided evenly to cells in the microfluidic channel.

### SUMMARY

Accordingly, there is a need for microfluidic devices for cell culture that address the above-discussed challenges and restrictions. The microfluidic devices described in this application address the above-discussed challenges and restrictions. In addition, the microfluidic devices described in this application address additional challenges and restrictions associated with conventional microfluidic devices, some of which are described below.

In accordance with some embodiments, a microfluidic device has a microfluidic channel which is partially enclosed (e.g., open at least on its top or one side along a portion of the length of the microfluidic channel).

In accordance with some embodiments, a device includes a substrate having a top surface and a bottom surface opposite to the top surface; a first microfluidic channel defined on the substrate; and a second microfluidic channel, distinct from the first microfluidic channel, defined on the substrate and in contact with, and substantially parallel to, the first microfluidic channel.

In accordance with some embodiments, a method for forming an endothelialized microchannel includes injecting a first solution into a first channel of a microfluidic device; and injecting a second solution into a second channel of the microfluidic device that is communicable with the first channel for forming an endothelialized microchannel.

In accordance with some embodiments, a method for forming a vascularized tumor spheroid includes injecting a first solution into a first channel of a microfluidic device. The first solution includes a tumor spheroid. The method also includes injecting a second solution into a second channel of the microfluidic device that is communicable with the first channel for forming a vascularized tumor spheroid.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A illustrates a method for making a device with a fluidic channel in accordance with some embodiments.

FIG. 1B illustrates components used for making a device array in accordance with some embodiments.

FIGS. 2A and 2B are top and bottom views of the device shown in FIG. 1A in accordance with some embodiments.

FIG. 2C is a cross-sectional view of the device shown in FIG. 1A in accordance with some embodiments.

FIG. 2D is a cutout view of the device shown in FIG. 1A in accordance with some embodiments.

FIG. 3 illustrates a method of using the device shown in FIG. 1A in accordance with some embodiments.

FIGS. 4A and 4B illustrate filling a first channel of the device shown in FIG. 1A in accordance with some embodiments.

FIGS. 5A-5C illustrate conditions for successfully filling the first channel of the device shown in FIG. 1A in accordance with some embodiments.

FIGS. 6A and 6B illustrate filling a second channel of the device shown in FIG. 1A in accordance with some embodiments.

FIGS. 7A-7C illustrate conditions for successfully filling the second channel of the device shown in FIG. 1A in accordance with some embodiments.

FIGS. 8A and 8B illustrate conditions for successfully filling the first channel and the second channel of the device shown in FIG. 1A in accordance with some embodiments.

FIG. 9A illustrates forming a perfusable vascular network with a spheroid in accordance with some embodiments.

FIG. 9B illustrates forming a perfusable vascular network without a spheroid in accordance with some embodiments.

FIG. 10 illustrates perfusable vascular networks formed under different conditions in accordance with some embodiments.

FIG. 11 illustrates vascular networks formed by using SW620 cells in accordance with some embodiments.

FIG. 12 illustrates neural networks formed by using primary neural cells in accordance with some embodiments.

FIG. 13 illustrates vascular networks formed by using human brain microvascular endothelial cells (HBMECs) in accordance with some embodiments.

FIG. 14 illustrates angiogenesis on a device shown in FIG. 1A in accordance with some embodiments.

FIG. 15 illustrates angiogenesis with a spheroid in accordance with some embodiments.

FIG. 16 illustrates formation of a vascularized tumor spheroid in accordance with some embodiments.

FIG. 17A illustrates a device with a fluidic channel in accordance with some embodiments.

FIG. 17B illustrates a cross-sectional view of the device shown in FIG. 17A with liquid patterning in accordance with some embodiments.

FIG. 17C illustrates a bottom view of the device shown in FIG. 17A with liquid patterning in accordance with some embodiments.

FIG. 18 illustrates conditions for successfully filling the second channel of the device shown in FIG. 17A in accordance with some embodiments.

FIG. 19 illustrates endothelialized microchannels formed by using the device shown in FIG. 17A.

FIG. 20 illustrates perfusable blood vessel networks in accordance with some embodiments.

FIG. 21 illustrates perfusable blood vessel networks in accordance with some embodiments.

FIG. 22 shows a comparison of a tissue model with reduced endothelialization and a tissue model with endothelialized microchannels.

FIG. 23 shows a comparison of a tissue model without endothelialization and a tissue model with endothelialized microchannels.

FIG. 24 shows formation of a perfusable blood vessel network in accordance with some embodiments.

FIG. 25 shows formation of a vascularized micro-tumor tissue in accordance with some embodiments.

FIG. 26 shows a vascularized cancer spheroid in accordance with some embodiments.

FIG. 27 shows formation of vascularized cancer spheroids in accordance with some embodiments.

FIG. 28 shows collection of a tissue in accordance with some embodiments.

FIG. 29 shows angiogenesis in a tissue grown in a fluidic device in accordance with some embodiments.

FIG. 30 shows angiogenesis in a tissue grown in a fluidic device in accordance with some embodiments.

FIG. 31 is a flow diagram illustrating a method for forming an endothelialized microchannel in accordance with some embodiments.

FIG. 32 is a flow diagram illustrating a method for forming a vascularized spheroid in accordance with some embodiments.

FIG. 33 is a flow diagram illustrating a method for forming a vascularized organoid in accordance with some embodiments.

### DETAILED DESCRIPTION

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

The “microfluidic channel” used herein refers to a path of fluid flow. In some cases, a fluid path defines a space in which cells or tissue is cultured and which is open at one or more sides (e.g., one lateral side, two lateral sides, three lateral sides, or four lateral sides) to be connected with another flow path or chamber so as to allow exchange of a culture medium and a fluid between adjacent fluids or chambers. In some embodiments, a microfluidic channel needs not be enclosed on two or more lateral sides.

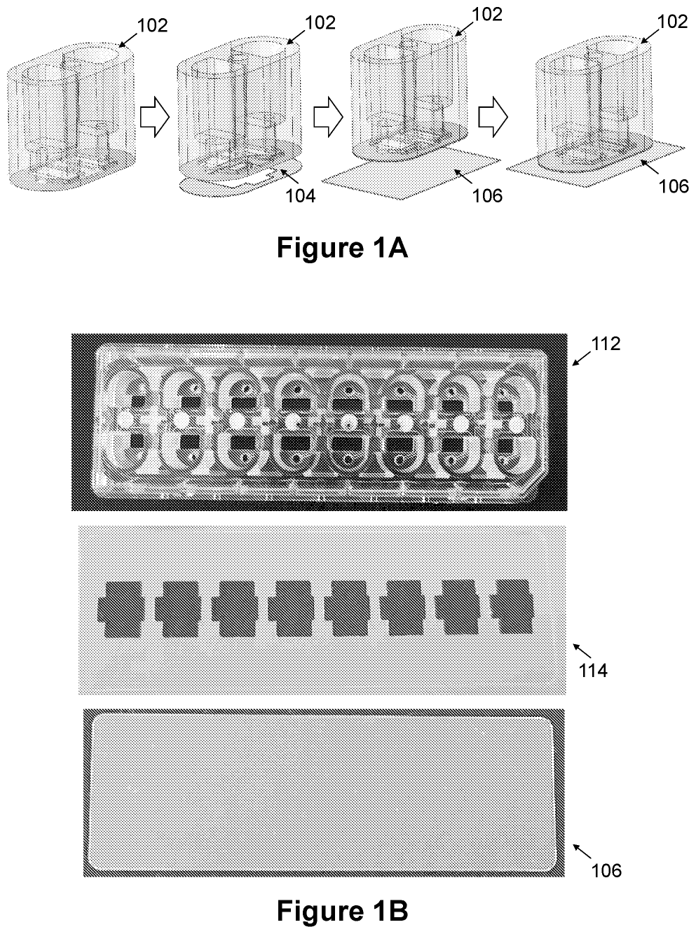

FIG. 1A illustrates a method for making a device with a fluidic channel in accordance with some embodiments.

The method includes obtaining a body 102. In some embodiments, the body 102 is made of a plastic material (e.g., polyethylene, polystyrene, polyvinyl chloride, polypropylene, polycarbonate, etc.). In some embodiments, the body 102 is made by using molding (e.g., injection molding, compression molding, insertion molding, etc.).

In some embodiments, the method includes attaching the body 102 to a sealer 104. In some embodiments, the sealer includes an adhesive layer (e.g., pressure sensitive adhesive tape).

The method includes attaching the body 102 to a substrate 106 (e.g., using the sealer 104) to form a fluidic device. In some embodiments, the substrate 106 is made of glass or a plastic material.

As shown in FIG. 1A, in some embodiments, a through-hole (or a cutout) is defined in the sealer 104 so that at least a portion of a liquid in the body 102 may contact directly with the substrate 106.

FIG. 1B illustrates components used for making a device array in accordance with some embodiments. Although FIG. 1A illustrates a method of making a device with a single culture well, it is possible to make an array of such devices concurrently by using a body 112 that defines multiple chambers for multiple culture wells, a sealer 114 with multiple through-holes (or cutouts), and a substrate 106.

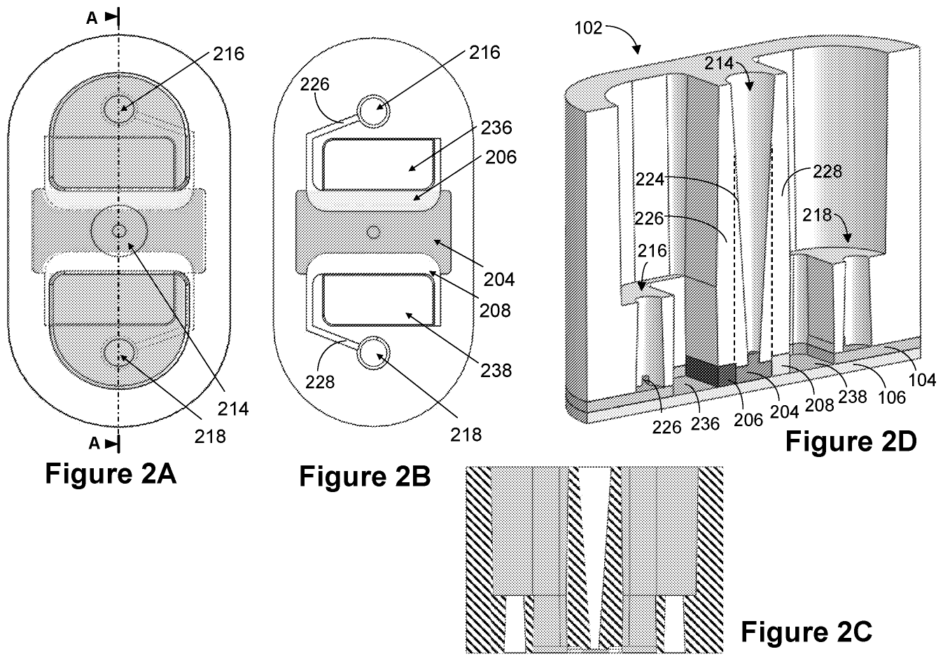

FIG. 2A shows a top view of the device shown in FIG. 1A in accordance with some embodiments. Shown in FIG. 2A are injection holes 214, 216, and 218. Injection hole 214 is connected to a first channel 204 (e.g., liquid provided into injection hole 214 fills first channel 204), injection hole 216 is connected to a second channel 206 (e.g., liquid provided into injection hole 216 fills second channel 206), and injection hole 218 is connected to a third channel 208 (e.g., liquid provided into injection hole 218 fills third channel 208). Also shown in FIG. 2A is line AA from which the cross-sectional view of FIG. 2C is taken.

FIG. 2B shows a bottom view of the device shown in FIG. 1A in accordance with some embodiments. Shown in FIG. 2B are first channel 204, channel 226 for routing liquid provided into injection hole 216 to second channel 206, and channel 228 for routing liquid provided into injection hole 218 to third channel 208. Also shown in FIG. 2B are reservoirs 236 and 238.

FIG. 2D is a cutout view of the device shown in FIG. 1A in accordance with some embodiments. The cutout view shows the connection of injection hole 214 to first channel 204, channel 226 connecting injection hole 216 to second channel 206, injection hole 218 connected to third channel 208, and reservoirs 236 and 238. As shown in FIG. 2D, injection hole 214 may define a reservoir for first liquid to be provided to first channel 204, injection hole 216 may define (along with a portion of sealer 104) a reservoir for second liquid to be provided to second channel 206, and injection hole 218 may define (along with a portion of sealer 104) a reservoir for third liquid to be provided to third channel 208.

In some embodiments, injection hole 214 is defined at least partially in beam 224. In FIG. 2D, beam 224 is separated from substrate 106 and first channel 204 is defined between beam 224 and substrate 106.

Located adjacent to beam 224 are beam 226 and beam 228. In FIG. 2D, beam 226 is in contact with beam 224 and beam 228 is in contact with beam 224. In some embodiments, beam 224 is integrally formed with beam 226 and beam 228. In FIG. 2D, beam 226 is separated from substrate 106 and second channel 206 is defined between beam 226 and substrate 106. In FIG. 2D, beam 228 is separated from substrate 106 and third channel 208 is defined between beam 228 and substrate 106.

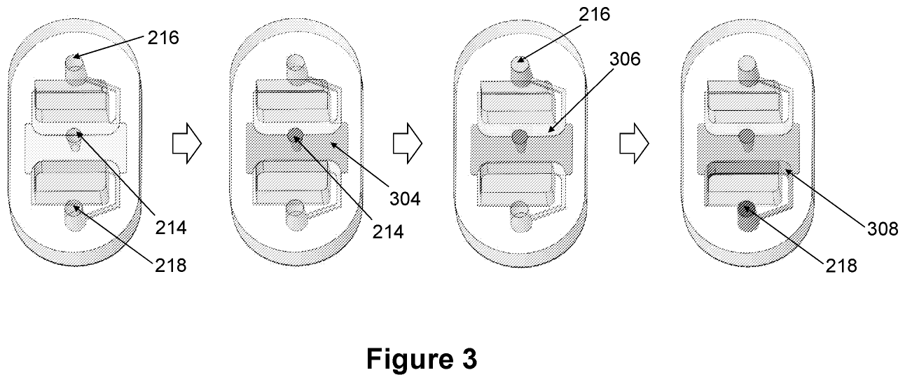

FIG. 3 illustrates a method of using the device shown in FIG. 1A in accordance with some embodiments. First liquid 304 is provided into injection hole 214 so that first liquid 304 fills first channel 204. Subsequent to providing first liquid 304 into injection hole 214, second liquid 306 is provided into injection hole 216 so that second liquid 306 fills second channel 206 (through channel 226) and third liquid 308 is provided into injection hole 218 so that third liquid 308 fills third channel 208. In some embodiments, second liquid 306 is provided into injection hole 216 concurrently with providing third liquid 308 into injection hole 218. In some embodiments, second liquid 306 is provided into injection hole 216 before or after providing third liquid 308 into injection hole 218.

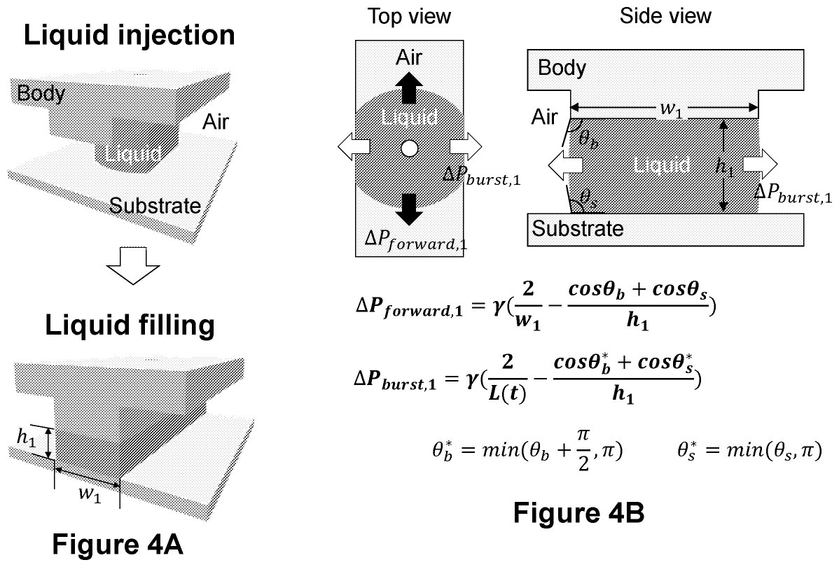

FIGS. 4A and 4B illustrate filling first channel 204 of the device shown in FIG. 1A in accordance with some embodiments. In some cases, the first liquid provided into first channel 204 (via injection hole 214) spread and fill first channel 204 (e.g., by surface tension) when the following conditions are met:

$$Δ {P}_{forward , 1} - Δ {P}_{burst , 1} < 0$$

$$where$$

$$Δ {P}_{forward , 1} = γ ( \frac{2}{{w}_{1}} - \frac{cos {θ}_{b} + cos {θ}_{s}}{{h}_{1}} )$$

$$Δ {P}_{burst , 1} = γ ( \frac{2}{L ( t )} - \frac{cos {θ}_{b}^{*} + cos {θ}_{s}^{*}}{{h}_{1}} )$$

$${θ}_{b}^{*} = min ( {θ}_{b} + \frac{π}{2} , π )$$

$${θ}_{s}^{*} = min ( {θ}_{s} , π )$$

w₁ is a width of first channel 204, h₁ is a height of first channel 204, γ is a surface tension of the first liquid, body contact angle θ_{b} is an advance contact angle between the first liquid and body 102, substrate contact angle θₛ is an advancing contact angle between the first liquid and substrate 106, and L(t) is the length of a volume occupied by the first fluid within first channel 204 at time t. In these equations, L(t) may be called L₁(t).

Examples of the advancing contact angle are as follows:

| | | UV-curable Acrylate Resin |
|---|---|---|
| Body Material | Polystyrene | (e.g., MED-AMB-10) |
| Advancing | 106.67° | 93.99° |
| Contact Angle | | |

| Substrate | | Pressure Sensitive | | Fibrin |
|---|---|---|---|---|
| Material | Glass | Adhesive Film | Polycarbonate | Gel |
| Advancing | 22.34° | 121.02° | 97.23° | 130° |
| Contact Angle | | | | |

In some embodiments, it is desirable to have ΔP_{burst,1} greater than ΔP_{forward,1}. (e.g., a pressure difference ΔP₁=ΔP_{burst,1}−ΔP_{forward,1}>0). In some configurations, it may be beneficial to maximize ΔP₁ (or provide ΔP₁ greater than a predefined threshold).

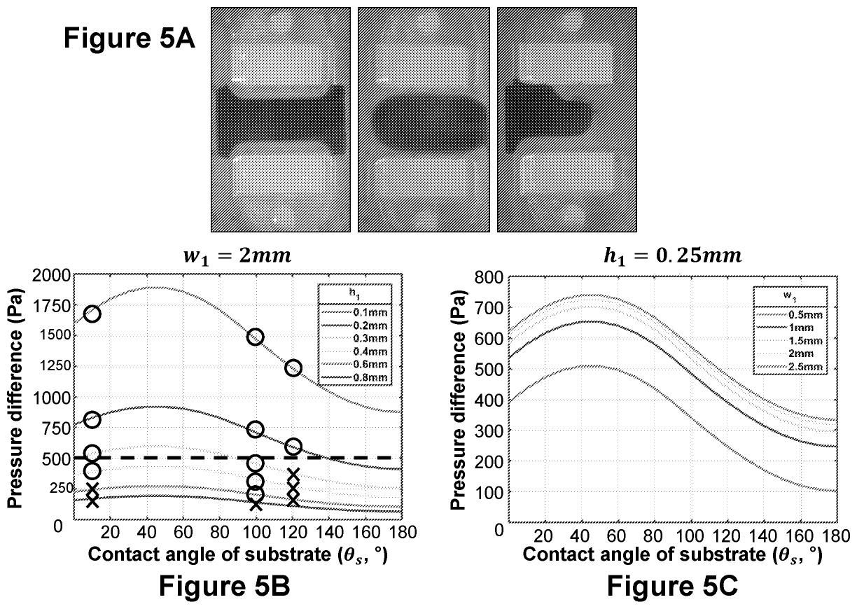

FIGS. 5A-5C illustrate conditions for successfully filling first channel 204 of the device shown in FIG. 1A in accordance with some embodiments. FIG. 5B shows a pressure difference ΔP₁ as a function of a contact angle of substrate θₛ in degrees for a liquid having surface tension γ of 72 mN/m (e.g., water at 25° C.) and a body contact angle of 70° (advancing contact angle of water in contact with a body made of polystyrene with surface treatment) in a first channel having a width w₁ of 2 mm. As shown in FIG. 5B, the pressure difference ΔP₁ of 500 Pa or greater led to successful filling of the first channel with the first liquid (as shown in FIG. 5A, left side, which was obtained with a first channel having w₁ of 2 mm and h₁ of 0.3 mm for a liquid having θₛ of 0° and θ_{b} of 70°). The conditions in which the pressure difference ΔP₁ was less than 500 Pa could lead to underfilling of the first channel (as shown in FIG. 5A, middle, which was obtained with a first channel having w₁ of 2 mm and h₁ of 0.7 mm for a liquid having θₛ of 0° and θ_{b} of 70°) or spilling into an adjacent second or third channel (as shown in FIG. 5A, right side, which was obtained with a first channel having w₁ of 2 mm and h₁ of 0.3 mm for a liquid having θₛ of 120° and θ_{b} of 70°). FIG. 5C shows a pressure difference ΔP₁ as a function of a contact angle of substrate θₛ in degrees for a liquid having surface tension γ of 72 mN/m (e.g., water at 25° C.) and a body contact angle of 70° (advancing contact angle of water in contact with a body made of polystyrene with surface treatment) in a first channel having a height h₁ of 0.25 mm.

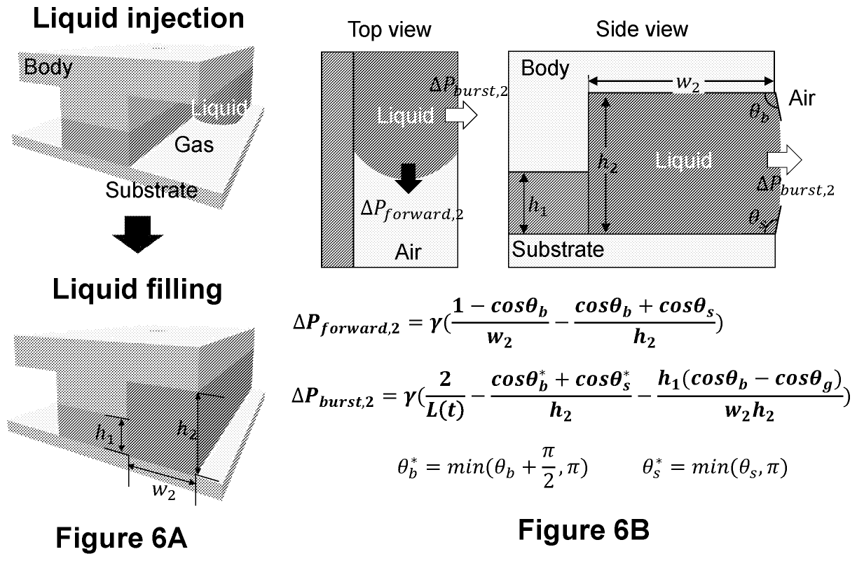

FIGS. 6A and 6B illustrate filling a second channel 206 of the device shown in FIG. 1A in accordance with some embodiments. In some cases, the second liquid provided into second channel 206 (via injection hole 216) spread and fill second channel 206 (e.g., by surface tension) when the following conditions are met:

$$Δ {P}_{forward , 2} - Δ {P}_{burst , 2} < 0$$

$$where$$

$$Δ {P}_{forward , 2} = γ ( \frac{1 - cos {θ}_{b}}{{w}_{2}} - \frac{cos {θ}_{b} + cos {θ}_{s}}{{h}_{2}} )$$

$$Δ {P}_{burst , 2} = γ ( \frac{2}{L ( t )} - \frac{cos {θ}_{b}^{*} + cos {θ}_{s}^{*}}{{h}_{2}} - \frac{{h}_{1} ( cos {θ}_{b} - cos {θ}_{g} )}{{w}_{2} {h}_{2}} )$$

$${θ}_{b}^{*} = min ( {θ}_{b} + \frac{π}{2} , π )$$

$${θ}_{s}^{*} = min ( {θ}_{s} , π )$$

w₂ is a width of second channel 206, h₂ is a height of second channel 206, γ is a surface tension of the second liquid, body contact angle θ_{b} is an advance contact angle between the second liquid and body 102, substrate contact angle θₛ is an advancing contact angle between the second liquid and substrate 106, and L(t) is the length of a volume occupied by the second fluid within second channel 206 at time t. In these equations, L(t) may be called L₂(t).

In some embodiments, it is desirable to have ΔP_{burst,2} greater than ΔP_{forward,2}. (e.g., a pressure difference ΔP₂=ΔP_{burst,2}−ΔP_{forward,2}>0). In some configurations, it may be beneficial to maximize ΔP₂ (or provide ΔP₂ greater than a predefined threshold).

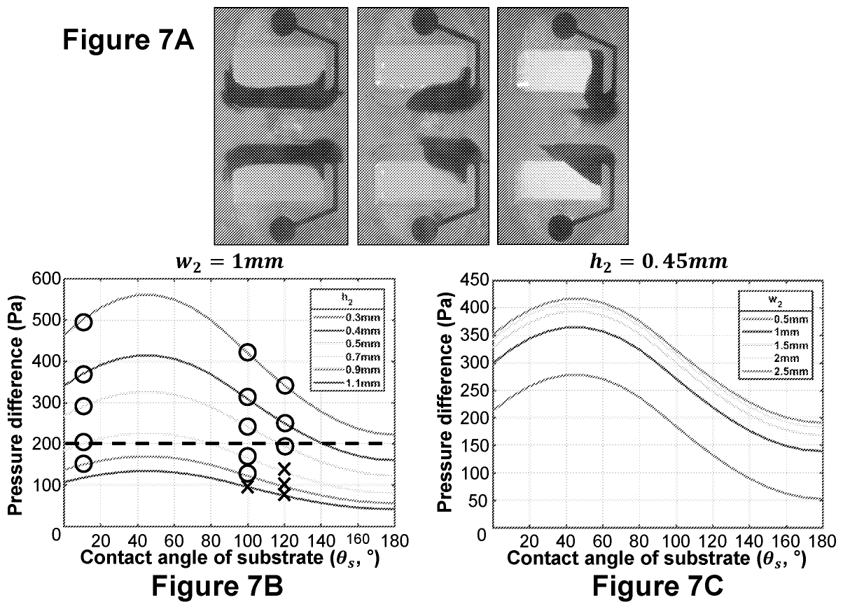

FIGS. 7A-7C illustrate conditions for successfully filling second channel 206 of the device shown in FIG. 1A in accordance with some embodiments.

FIG. 7B shows a pressure difference ΔP₂ as a function of a contact angle of substrate θₛ in degrees for a liquid having surface tension γ of 72 mN/m (e.g., water at 25° C.) and a body contact angle of 70° (advancing contact angle of water in contact with a body made of polystyrene with surface treatment) in a second channel having a width w₂ of 1 mm. As shown in FIG. 7B, the pressure difference ΔP₂ of 200 Pa or greater led to successful filling of the second channel with the second liquid (as shown in FIG. 7A, left side, which was obtained with a second channel having w₂ of 1 mm and h₂ of 0.6 mm adjacent to a first channel having h₁ of 0.25 mm for a liquid having θₛ of 0° and θ_{b} of 70°). The conditions in which the pressure difference ΔP₂ was less than 200 Pa could lead to underfilling of the second channel (as shown in FIG. 7A, middle, which was obtained with a second channel having w₂ of 1 mm and h₂ of 1 mm adjacent to a first channel having h₁ of 0.25 mm for a liquid having θₛ of 0° and θ_{b} of 70°) or spilling into the adjacent first channel (as shown in FIG. 7A, right side, which was obtained with a second channel having w₂ of 1 mm and h₂ of 0.6 mm adjacent to a first channel having h₁ of 0.25 mm for a liquid having θₛ of 120° and θ_{b} of 70°). FIG. 7C shows a pressure difference ΔP₂ as a function of a contact angle of substrate θₛ in degrees for a liquid having surface tension γ of 72 mN/m (e.g., water at 25° C.) and a body contact angle of 70° (advancing contact angle of water in contact with a body made of polystyrene with surface treatment) in a second channel having a height h₂ of 0.45 mm.

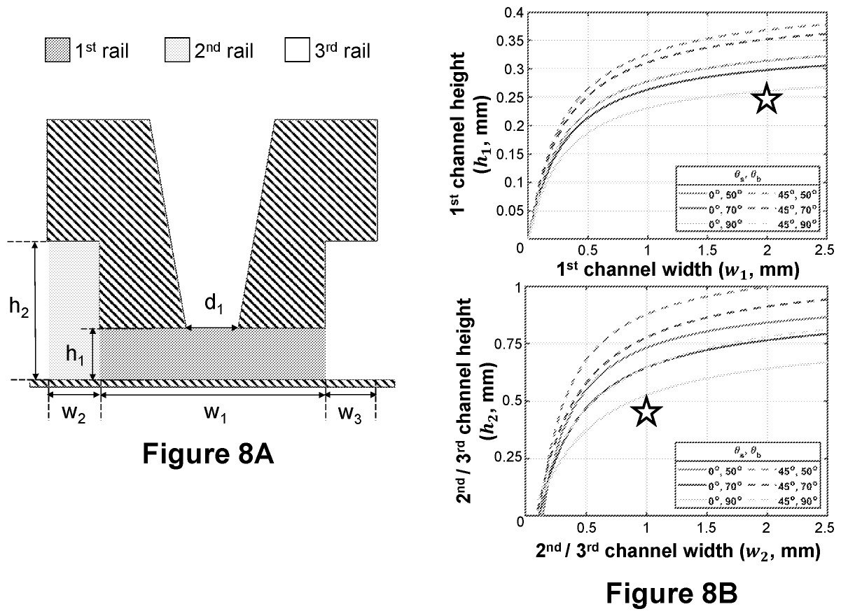

FIGS. 8A and 8B illustrate conditions for successfully filling the first channel and the second channel of the device shown in FIG. 1A in accordance with some embodiments. FIG. 8A shows that the device has a first channel with a width w₁ and a height h₁, a second channel with a width w₂ and a height h₂, and a third channel with a width w₃ and a height h₃. In some embodiments, w₂ and w₃ are identical. In some embodiments, w₂ is distinct from w₃. In some embodiments, h₂ and h₃ are identical. In some embodiments, h₂ is distinct from h₃. In some embodiments, the injection hole 214 has a diameter of d₁.

The top chart of FIG. 8B shows the height h₁ that can be selected for a given width w₁ (and the body contact angle θ_{b} and the substrate contact angle θₛ). For example, a first channel having a height h₁ below a curve selected for given body contact angle θ_{b} and substrate contact angle θₛ can allow the first liquid to successfully fill the first channel. Similarly, the bottom chart of FIG. 8B shows the height h₂ that can be selected for a given width w₂ (and the body contact angle θ_{b} and the substrate contact angle θₛ). For example, a second channel having a height h₂ below a curve selected for given body contact angle θ_{b} and substrate contact angle θₛ can allow the second liquid to successfully fill the second channel.

The conditions described with respect to FIGS. 6A-6B, 7A-7C, and 8A-8B for filling a second channel may apply similarly to filling a third channel of the device shown in FIG. 1A. For brevity, such details are not repeated herein.

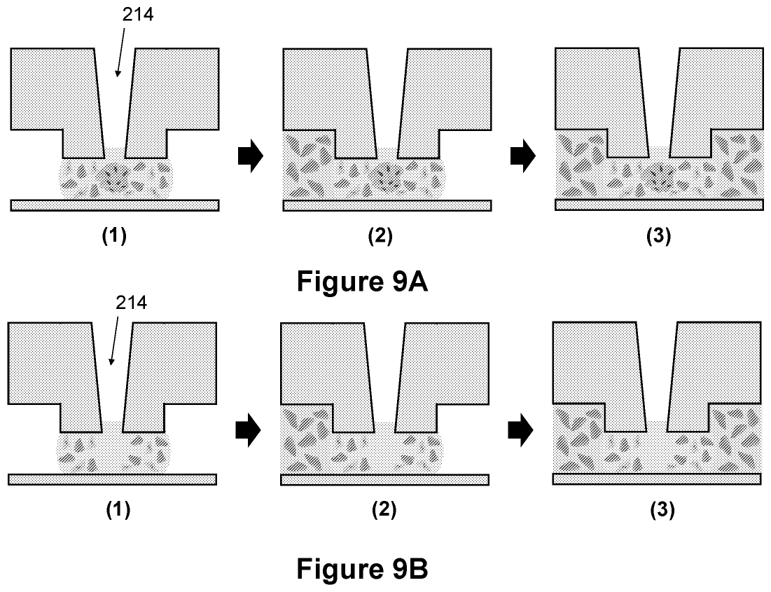

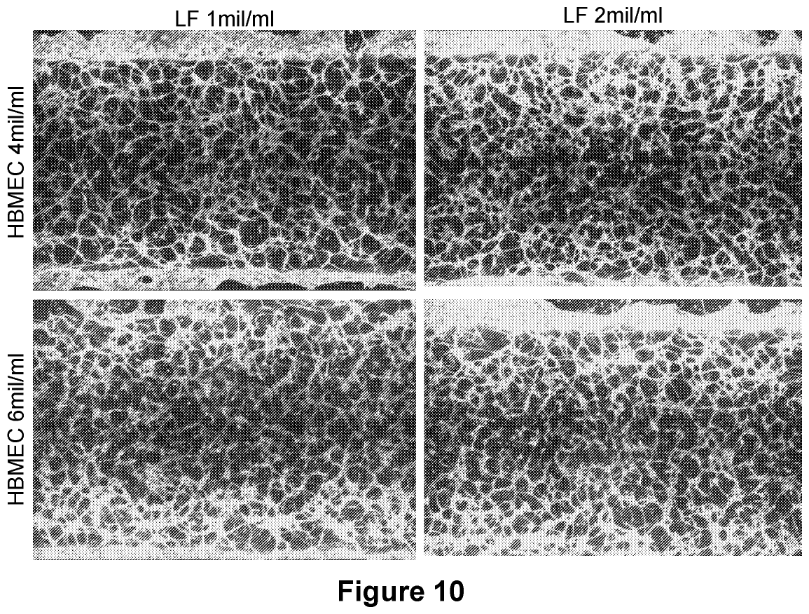

The devices described herein (including the device shown in FIG. 1A) can be used in various applications. For example, FIG. 9A illustrates forming a perfusable vascular network with a spheroid in accordance with some embodiments. For forming a perfusable vascular network with a spheroid, a tumor spheroid mixed with lung fibroblasts (LF) and endothelial cells (EC) is injected into the first channel through the injection hole 214. Subsequently, a mixture containing endothelial cells is added to the second channel and the third channel so that a perfusable vascular network can be formed.

FIG. 9B illustrates forming a perfusable vascular network without a spheroid in accordance with some embodiments. For example, a mixture of lung fibroblasts (LF) and endothelial cells (EC) is injected into the first channel through the injection hole 214. The mixture shown in FIG. 9B does not include a spheroid. Subsequently, a mixture containing endothelial cells is added to the second channel and the third channel so that a perfusable vascular network can be formed.

FIG. 10 illustrates perfusable vascular networks formed under different conditions in accordance with some embodiments. Shown in FIG. 10 are perfusable vascular networks formed with a mixture of human brain microvascular endothelial cells (HBMEC) and lung fibroblasts (LF) where the concentration of HBMEC was either 4 mi/ml (million cells/mL) or 6 mi/ml and the concentration of LF was either 1 mi/ml or 2 mi/ml. These photographs show that perfusable vascular networks were successfully formed after incubation.

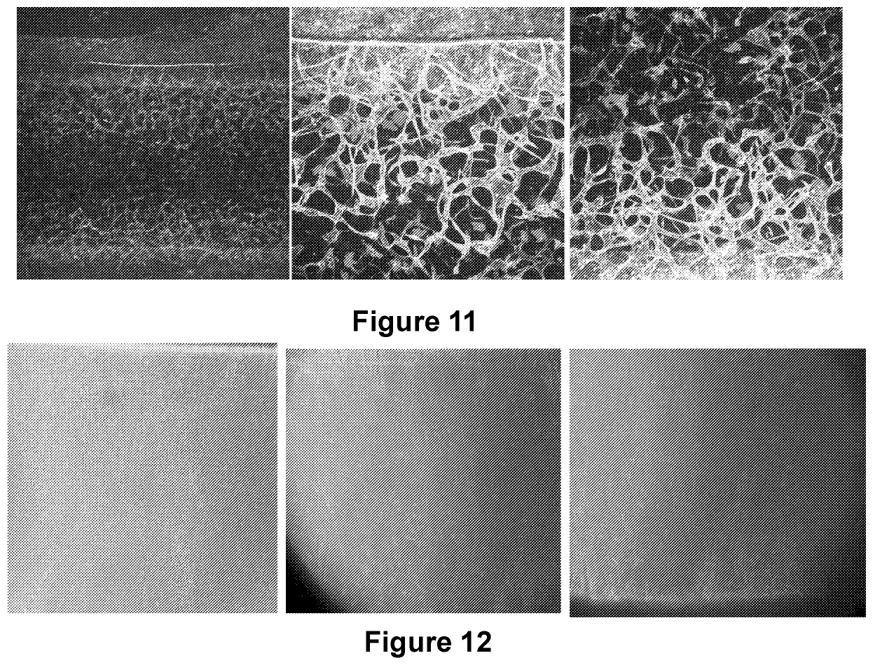

FIG. 11 illustrates vascular networks formed by using SW620 cells in accordance with some embodiments. SW620 cells are derived from colorectal adenocarcinoma cell line. A 6 μL mixture of SW620 cells at 0.2 mi/ml, human umbilical vein endothelial cells P5 (HUVECs) at 6 mi/ml, lung fibroblasts (LF) at 3 mi/ml, and fibrin gel 2.5 mg/ml was provided into the first channel and a 10 μL solution containing HUVECs P5 at 1 mi/ml was provided to the second and third channels. The cancer-vessel model formed from the mixture, after incubation, is shown in FIG. 11 (left). Enlarged views of the cancer-vessel model are also shown in FIG. 11 (middle—showing an interface between the first channel and the second channel; right—showing an interface between the first channel and the third channel).

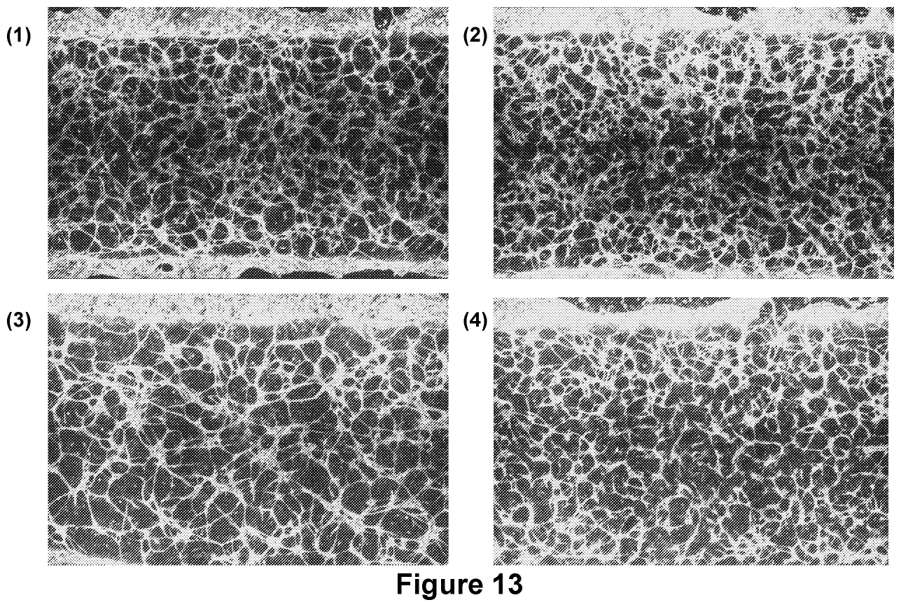

FIG. 12 illustrates neural networks formed by using primary neural cells in accordance with some embodiments. A 6 μL mixture of gelatinous protein (e.g., Matrigel) was provided into the first channel and a 10 μL solution containing primary neural cells at 8 mi/ml was provided to the second and third channels. Neural networks formed from the mixture, after incubation, are shown in FIG. 12.

FIG. 13 illustrates vascular networks formed by using human brain microvascular endothelial cells (HBMECs) in accordance with some embodiments. The vascular networks were formed using HBMECs under various conditions. Shown in FIG. 13 are (1) vascular networks formed using a 6 μL mixture of HBMEC P5 at 4 mi/ml, LF P6 at 1 mi/ml in fibrin gel at 2.5 mg/ml in the first channel and a 10 μL solution containing HBMEC P4 suspension at 1 mi/ml in the second and third channels, (2) vascular networks formed using a 6 μL mixture of HBMEC P5 at 4 mi/ml, LF P6 at 2 mi/ml in fibrin gel at 2.5 mg/ml in the first channel and a 10 μL solution containing HBMEC P4 suspension at 1 mi/ml in the second and third channels, (3) vascular networks formed using a 6 μL mixture of HBMEC P5 at 6 mi/ml, LF P6 at 1 mi/ml in fibrin gel at 2.5 mg/ml in the first channel and a 10 μL solution containing HBMEC P4 suspension at 1 mi/ml in the second and third channels, and (4) vascular networks formed using a 6 μL mixture of HBMEC P5 at 6 mi/ml, LF P6 at 2 mi/ml in fibrin gel at 2.5 mg/ml in the first channel and a 10 μL solution containing HBMEC P4 suspension at 1 mi/ml in the second and third channels.

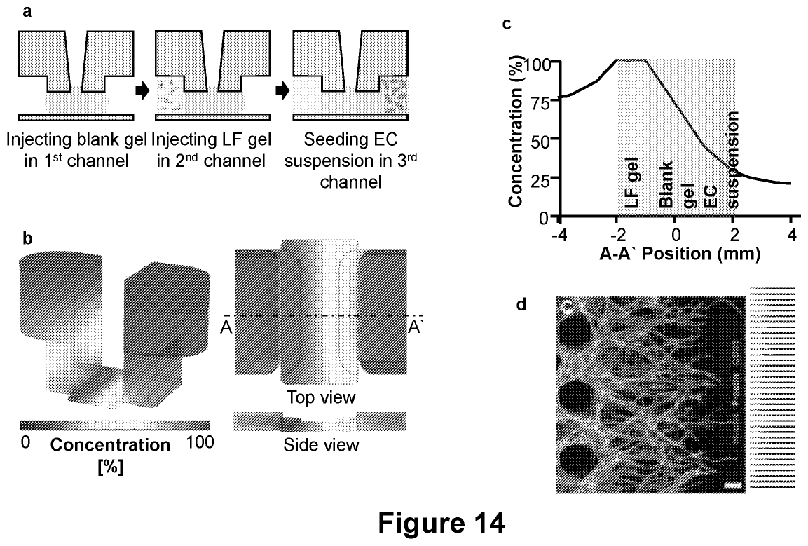

FIG. 14 illustrates angiogenesis on a device shown in FIG. 1A in accordance with some embodiments. For inducing angiogenesis, in some embodiments, as shown in section (a) of FIG. 14, a blank gel is provided to the first channel, and a gel containing lung fibroblasts is provided to the second channel, and a solution containing seeding endothelial cells is provided to the third channel. FIG. 14 also shows three-dimensional distribution of vascular endothelial growth factor (VEGF) in the device (section b) and distribution of VEGF molecules along the line AA′ shown in section b (section c). Section 4 of FIG. 14 shows vessels formed after 7 days of incubation.

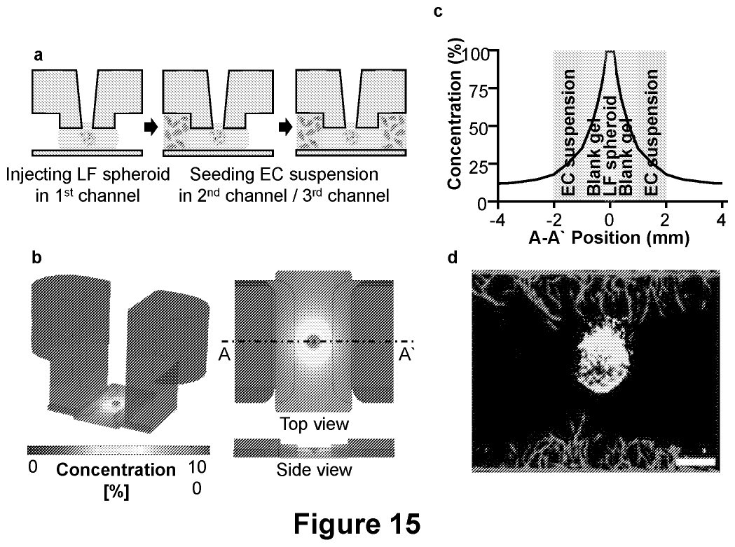

FIG. 15 illustrates angiogenesis with a spheroid (e.g., three-dimensional substantially spherical cellular aggregates) in accordance with some embodiments. For inducing angiogenesis with a spheroid, in some embodiments, as shown in section (a) of FIG. 15, a mixture of a spheroid and a blank gel is provided to the first channel, and a solution containing seeding endothelial cells is provided to the second and third channels. FIG. 15 also shows three-dimensional distribution of vascular endothelial growth factor (VEGF) in the device (section b) and distribution of VEGF molecules along the line AA′ shown in section b (section c). Section 4 of FIG. 15 shows vessels formed around the spheroid after 7 days of incubation. Although FIG. 15 shows an example using a spheroid, in some embodiments, an organoid (e.g., self-organized three-dimensional tissue cultures derived from stem cells) is used instead of a spheroid.

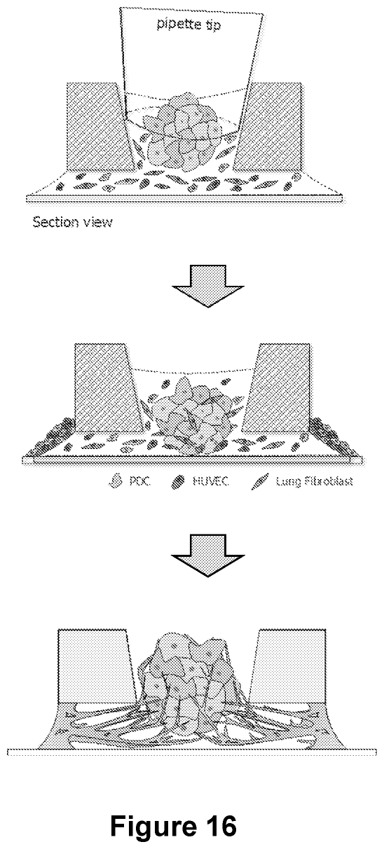

FIG. 16 illustrates formation of a vascularized tumor spheroid in accordance with some embodiments. In FIG. 16, a mixture of a spheroid (e.g., a tumor spheroid), HUVEC, lung fibroblasts, and hydrogel, such as fibrinogen, collagen, or protein mixture secreted by mouse sarcoma cells, is provided into the first channel. In some embodiments, the mixture also includes thrombin. Exposed interfaces (e.g., lateral interfaces) of the first solution are coated with endothelial cells. After incubation, vascular networks to the tumor spheroid are formed. The formed model (containing the vascularized tumor spheroid) may be used for studying the behavior of tumor in microenvironment. For example, the formed model may be used to screen pharmaceuticals or cells that may be effectively delivered to the tumor spheroid through the three-dimensional vascular networks.

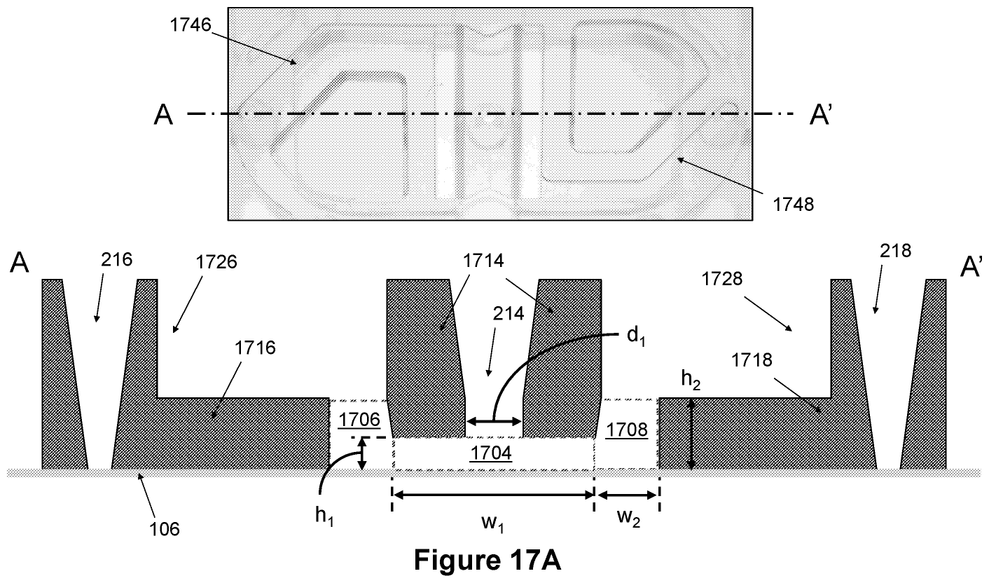

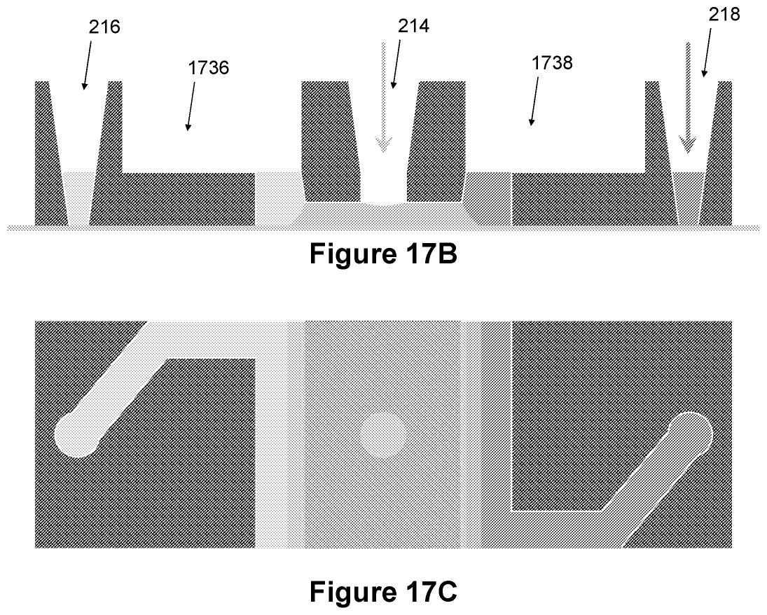

FIG. 17A illustrates a plan view of a device with a fluidic channel in accordance with some embodiments and a cross-section of the device along the line AA′. The device shown in FIG. 17A is similar to the device shown in FIG. 1A, except that the second and third channels in FIG. 17A are exposed on top, whereas the second and third channels in FIG. 1A are exposed laterally. In FIG. 17A, the device includes beam 1714 so that first channel 1704 is defined between beam 1714 and substrate 106. The device in FIG. 17A also includes beam 1716 so that second channel 1706 is defined between beam 1714 and beam 1716. In FIG. 17A, the device further includes beam 1718 so that third channel 1708 is defined between beam 1714 and beam 1718.

As shown in FIG. 17A, first channel 1704 has a width w₁, a height h₁, and a through-hole having a diameter d₁. In some embodiments, w₁ is between 0.1 mm and 10 mm, between 0.5 mm and 5 mm, between 1 mm and 3 mm, or between 1.5 mm and 2.5 mm. In some embodiments, h₁ is between 0.1 mm and 2 mm, between 0.1 mm and 1 mm, between 0.1 mm and 0.5 mm, or between 0.1 mm and 0.3 mm. In some embodiments, d₁ is between 0.1 mm and 1 mm, between 0.2 mm and 0.9 mm, between 0.3 mm and 0.8 mm, or between 0.4 mm and 0.7 mm. In some embodiments, d₁ is less than w₁.

Second channel 1706 (or third channel 1708) in FIG. 17A has a width of w₂ and a height of h₂. In some embodiments, w₂ is between 0.1 mm and 10 mm, between 0.5 mm and 5 mm, between 1 mm and 3 mm, or between 1.5 mm and 2.5 mm. In some embodiments, h₂ is between 0.1 mm and 10 mm, between 0.5 mm and 5 mm, between 1 mm and 3 mm, or between 1.5 mm and 2.5 mm. In some embodiments, h₂ is greater than h₁. Similar to the device shown in FIG. 1A, the device shown in FIG. 17A allows filling the second channel through the injection hole 216. However, the device shown in FIG. 17A allows a larger volume of liquid (e.g., 25 μL compared to 10 μL) to be injected into the injection hole 216. When the liquid contains endothelial cells, a monolayer of endothelial cells may be formed on an interface of the first liquid and the second liquid. Thus, the device shown in FIG. 17A can facilitate coating the liquid interface with endothelial cells, which, in turn, improves the reproducibility of experiments performed using the device shown in FIG. 17A and reduces the amount of endothelial cells needed for forming the endothelial cell layer.

In some embodiments, as shown in FIG. 17A, beam 1716 is included in (or a part of) a side structure 1726, which also defines the injection hole 216 and channel 1746 that allows the second liquid to flow from the injection hole 216 to second channel 1706. In some embodiments, as shown in FIG. 17A, beam 1718 is included in (or a part of) a side structure 1728, which also defines the injection hole 218 and channel 1748 that allows the second liquid to flow from the injection hole 218 to third channel 1708.

FIG. 17B illustrates a cross-sectional view of the device shown in FIG. 17A with liquid patterning in accordance with some embodiments, and FIG. 17C illustrates a bottom view of the device shown in FIG. 17A with liquid patterning in accordance with some embodiments. In some embodiments, the device is subjected to plasma treatment before injecting the liquids. In some embodiments, the plasma treatment makes certain surfaces within the device hydrophilic. A first liquid containing hydrogel (e.g., between 6 and 10 μL; however, a different volume may be used depending on the size of the device and the fluidic channels) is provided to the first channel through the injection hole 214. When the gelation of the hydrogel is performed in on a hydrophilic surface (e.g., a surface treated by plasma treatment), the interface of the hydrogel may have a concave surface as shown in FIG. 17B. After gelation of the hydrogel (e.g., ˜5 minutes for fibrinogen), second and third liquids are provided to the second channel and the third channel, respectively. In some embodiments, culture medium is provided to reservoirs 1736 and 1738, and the device is incubated.

For example, for constructing a vascularized tumor spheroid using the device shown in FIGS. 17A and 17B, a mixture of tumor spheroid, HUVECs 6.0 mi/ml, lung fibroblasts 3.0 mi/ml, fibrinogen, and thrombin is provided to the first channel through the injection hole 214 after the device has been subjected to plasma treatment. After the gelation of the fibrin gel (˜5 minutes), a solution containing endothelial cells in suspension is provided to the second channel and the third channel through the injection holes 216 and 218. Thereafter, 200-250 μL of culture medium is provided to reservoirs 1736 and 1738 and the device is incubated to form a vascularized tumor spheroid.

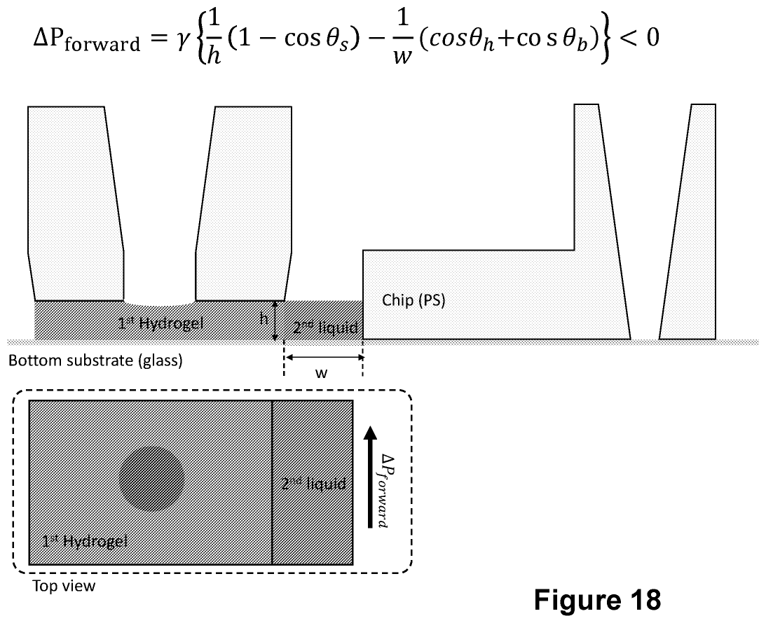

FIG. 18 illustrates conditions for successfully filling the second channel of the device shown in FIG. 17A in accordance with some embodiments. In some cases, the second liquid provided into second channel 206 (via injection hole 216) spread and fill second channel 206 (e.g., by surface tension) when the following condition is met:

$$Δ {P}_{forward} = γ \{ \frac{1}{h} ( 1 - cos {θ}_{s} ) - \frac{1}{w} ( cos {θ}_{h} + cos {θ}_{b} ) \} < 0$$

w is a width of second channel 206, h is a height of second channel 206, γ is a surface tension of the second liquid, body contact angle θ_{b} is an advance contact angle between the second liquid and body 102, substrate contact angle θₛ is an advancing contact angle between the second liquid and substrate 106, and gel contact angle θₕ is an advancing contact angle between the second liquid and the hydrogel in the first liquid (e.g., cured fibrinogen).

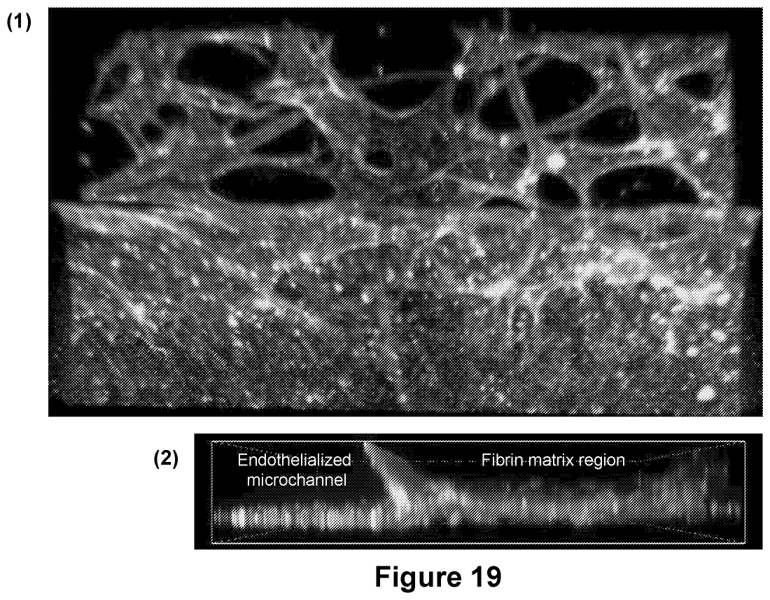

FIG. 19 illustrates endothelialized microchannels formed by using the device shown in FIG. 17A. The fluorescence shown in FIG. 19 is from lectin labeling endothelial cells. The image in FIG. 19 shows vascular networks and a monolayer of endothelial cells. The image also shows that endothelial cells in the vascular networks and the monolayer form anastomosis.

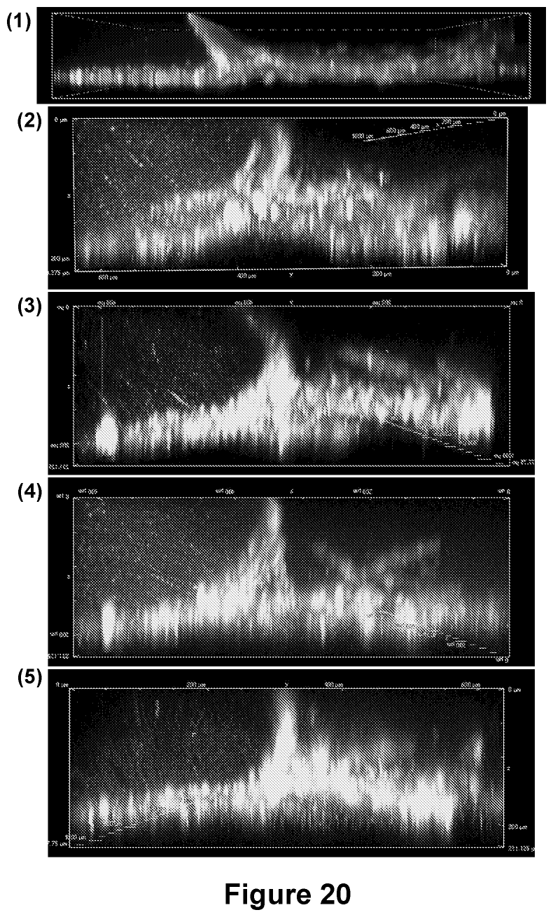

FIG. 20 illustrates perfusable blood vessel networks in accordance with some embodiments. The images in FIG. 20 show that HUVECs proliterate and cover the concave hydrogel interface. Although the curvature of the hydrogel interface changes based on the volume of the hydrogel provided to the first channel (1: open channel, 2:6 μL, 3:7 μL, 4:8 μL, and 5:10 μL), the monolayer of endothelial cells consistently covers the hydrogel interface regardless of the curvature of the hydrogel interface.

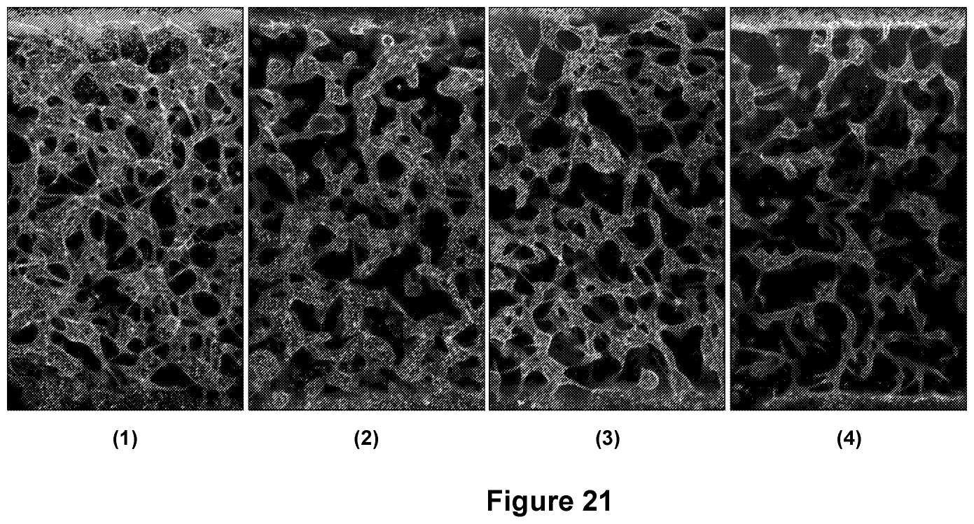

FIG. 21 illustrates perfusable blood vessel networks in accordance with some embodiments. The perfusable blood vessel networks formed by using a mixture of HUVECs, lung fibroblasts, and hydrogel (1: HUVEC 6 mi/ml, LF 6 mi/ml; 2: HUVEC 6 mi/ml, LF 3 mi/ml; 3: HUVEC 6 mi/ml, LF 2 mi/ml; and 4: HUVEC 6 mi/ml, LF 1 mi/ml) have different shapes, but all of the blood vessel networks allow delivery of 2 μm diameter microbeads therethrough.

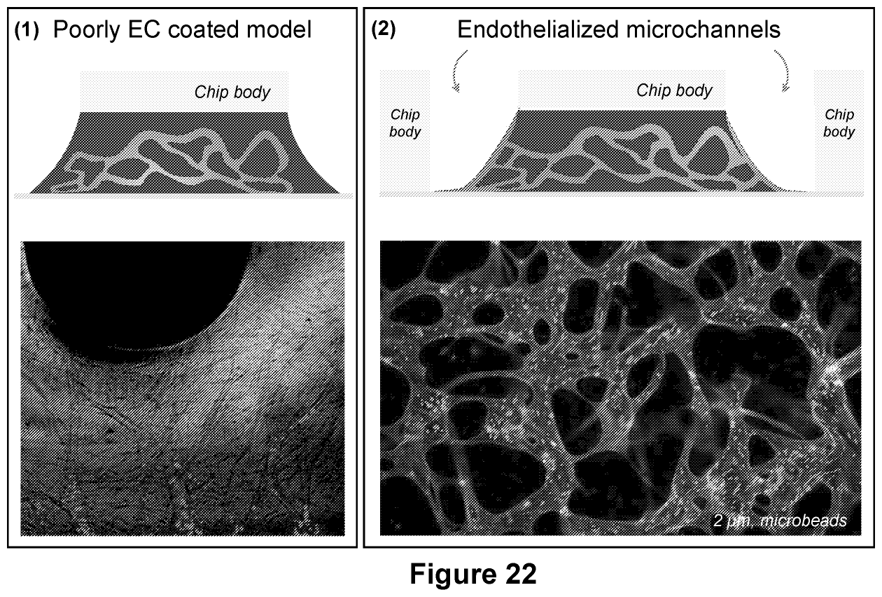

FIG. 22 shows a comparison of a tissue model with reduced endothelialization and a tissue model with endothelialized microchannels. Shown on the left side of FIG. 22 is a tissue model formed without an endothelial layer, which does not allow delivery of microbeads therethrough. In contrast, a tissue model shown on the right side of FIG. 22 has endothelial cell layers on the hydrogel interfaces, and the endothelial cell layers facilitate formation of vascular networks so that microbeads can be delivered therethrough.

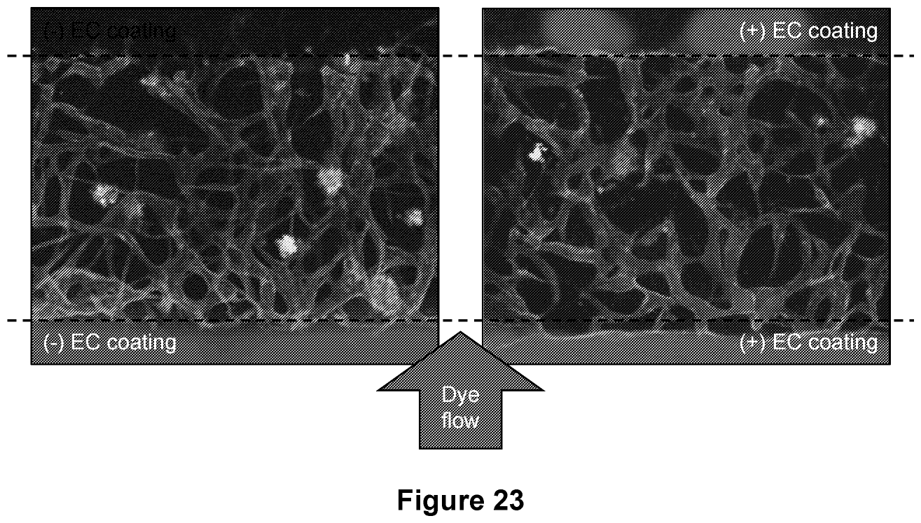

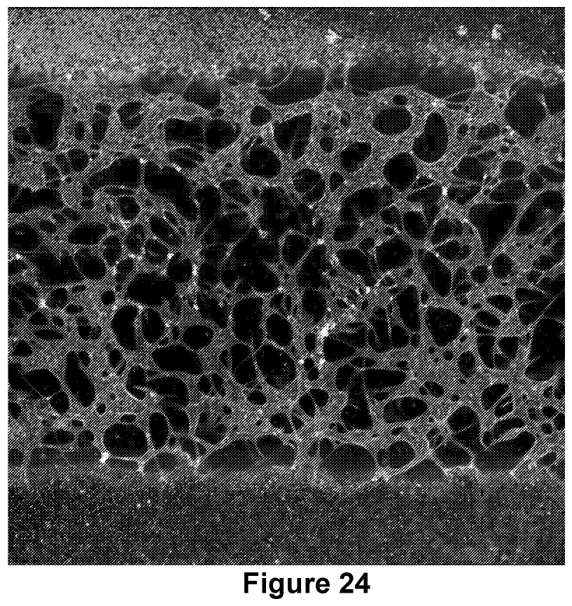

FIG. 23 shows a comparison of a tissue model without endothelialization and a tissue model with endothelialized microchannels. The vascularized spheroid formed using a layer of endothelial cells (shown on the right side) allows dye molecules to perfuse from the second channel (shown at the bottom) to the third channel (shown at the top) through the vascular networks formed in the first channel. In comparison, the vascularized spheroid formed without a layer of endothelial cells (shown on the left side) does not allow dye molecules to perfuse from the second channel (shown at the bottom) to the third channel (shown at the top).

FIG. 24 shows formation of a perfusable blood vessel network in accordance with some embodiments. The vascular network formed using a mixture of HUVEC at 6 mi/ml and LF at 3 mi/ml across a large area (e.g., over a distance of 2 mm) still allows perfusion of microbeads between the second channel and the third channel.

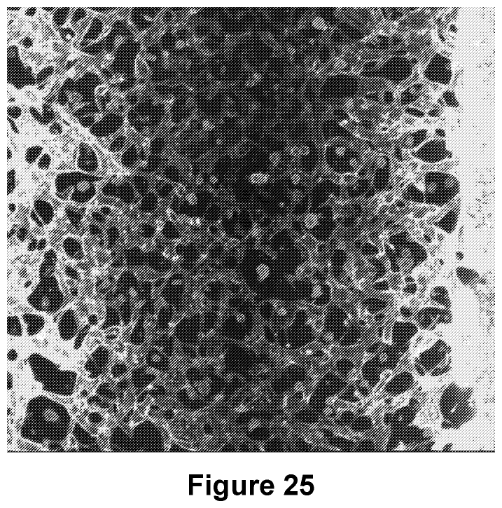

FIG. 25 shows formation of a vascularized micro-tumor tissue in accordance with some embodiments. The vascularized micro-tumor tissue was formed by incubating a mixture of HUVEC at 6 mi/ml, LF 3 mi/ml, and SW48 colon cancer cell at 0.2 mi/ml, and HUVEC-S at 0.85 mi/ml in the first channel for five days. The image shown in FIG. 25 was obtained by staining with lectin.

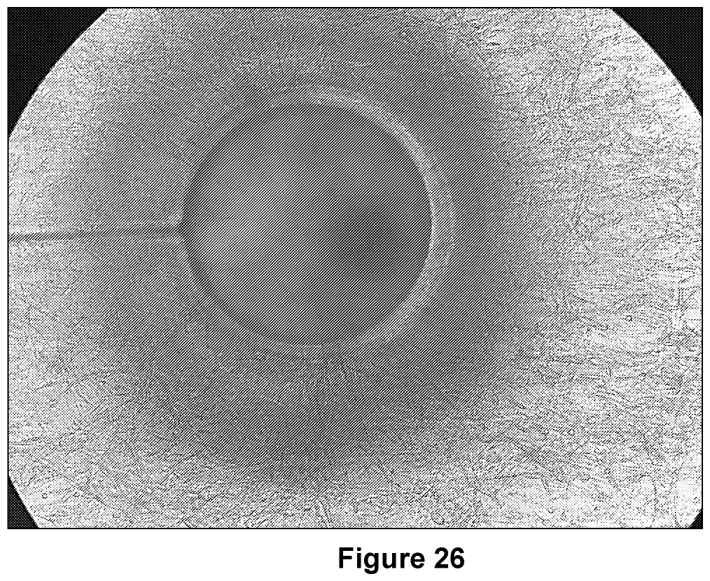

FIG. 26 shows a vascularized cancer spheroid in accordance with some embodiments. A PANC-1 cancer spheroid was mixed with LF and HUVECs at 1:9:4 ratio and provided to the first channel for incubation. The vascularized cancer spheroid formed after incubation for 8 days is shown in FIG. 26.

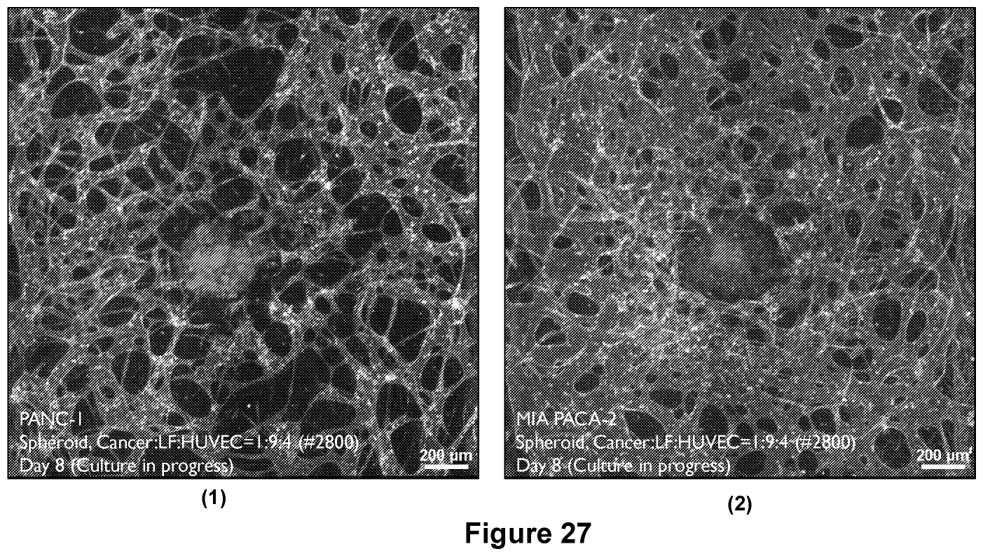

FIG. 27 shows formation of vascularized cancer spheroids in accordance with some embodiments (1: a vascularized PANC-1 spheroid; 2: a vascularized MIA PACA-2 spheroid). These vascularized cancer spheroids allow study of morphology of vascular networks based on types of cancer. In addition, the vascularized cancer spheroids allow study of biomarkers and phenotypes.

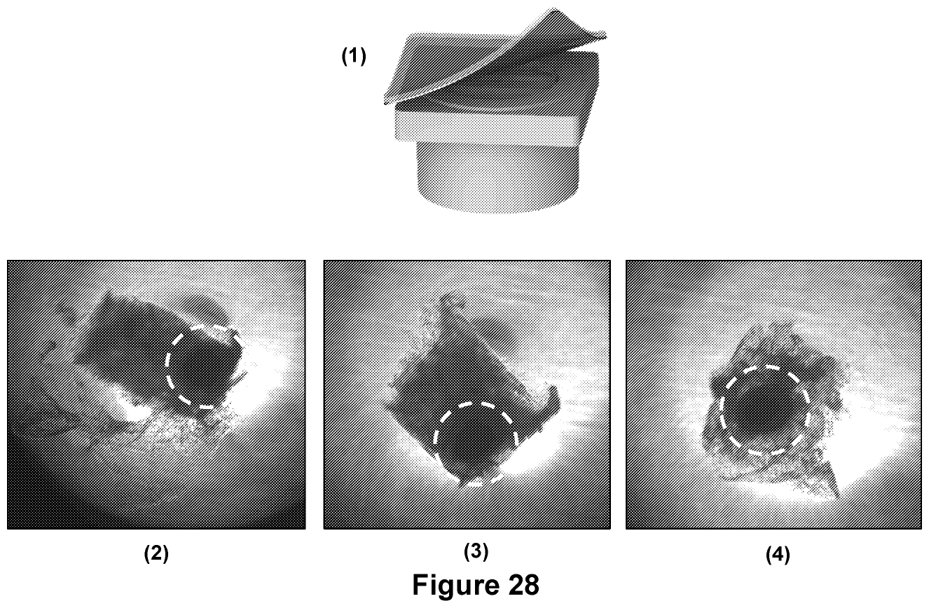

FIG. 28 shows collection of a tissue in accordance with some embodiments. The vascular networks formed as described herein need not be studied only within a device described herein. Instead, the tissue containing the vascular networks may be removed from the device so that the vascular networks may be used in other assays or subjected to other analysis methods. To facilitate the removal or collection of a tissue from the device, in some embodiments, a detachable substrate is used. After the vascular networks are formed, the detachable substrate is separated from the adhesive layer (e.g., the pressure sensitive adhesive tape or the body). In some embodiments, a portion of the detachable substrate in contact with the tissue is removed (e.g., by a biopsy punch). Thereafter, the detachable substrate, or a portion thereof, is placed in a buffer solution (e.g., phosphate buffer saline) to facilitate separation of the tissue from the detachable substrate.

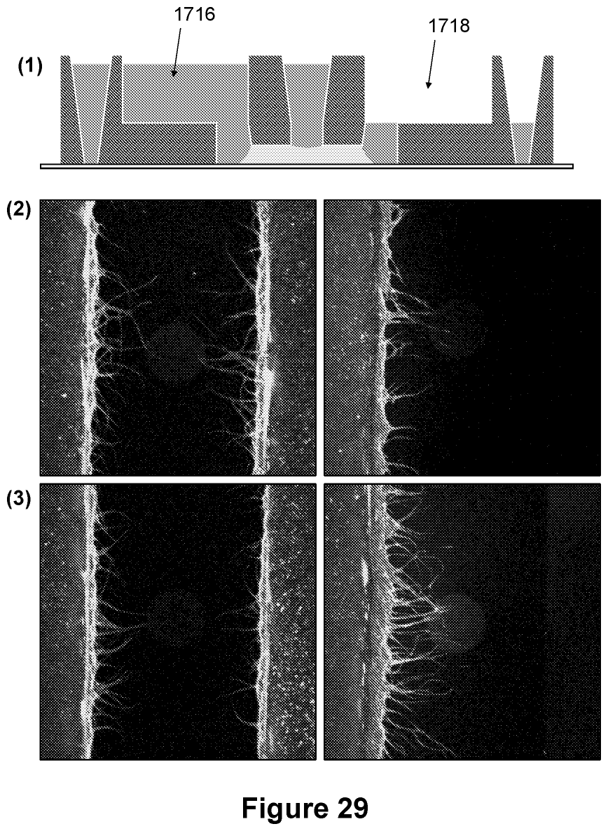

FIG. 29 shows angiogenesis in a tissue grown in a fluidic device in accordance with some embodiments. A mixture of lung fibroblasts and fibrin was provided to the first channel and a solution containing endothelial cells was provided to the second and third channels. As shown in section (1) of FIG. 29, different amounts of culture medium can be provided to the first reservoir 1736 and the second reservoir 1738 so that the pressure difference caused by the different levels of culture medium in the first reservoir 1736 and the second reservoir 1738 induces a flow through the hydrogel in the first channel. Section (2) of FIG. 29 shows endothelial cell sprouting when the same amounts of culture medium are provided to the first reservoir 1736 and the second reservoir 1738, and section (3) of FIG. 29 shows endothelial cell sprouting when the volumes of culture medium provided to the first reservoir 1736 and the second reservoir 1738 differ by 100 μL.

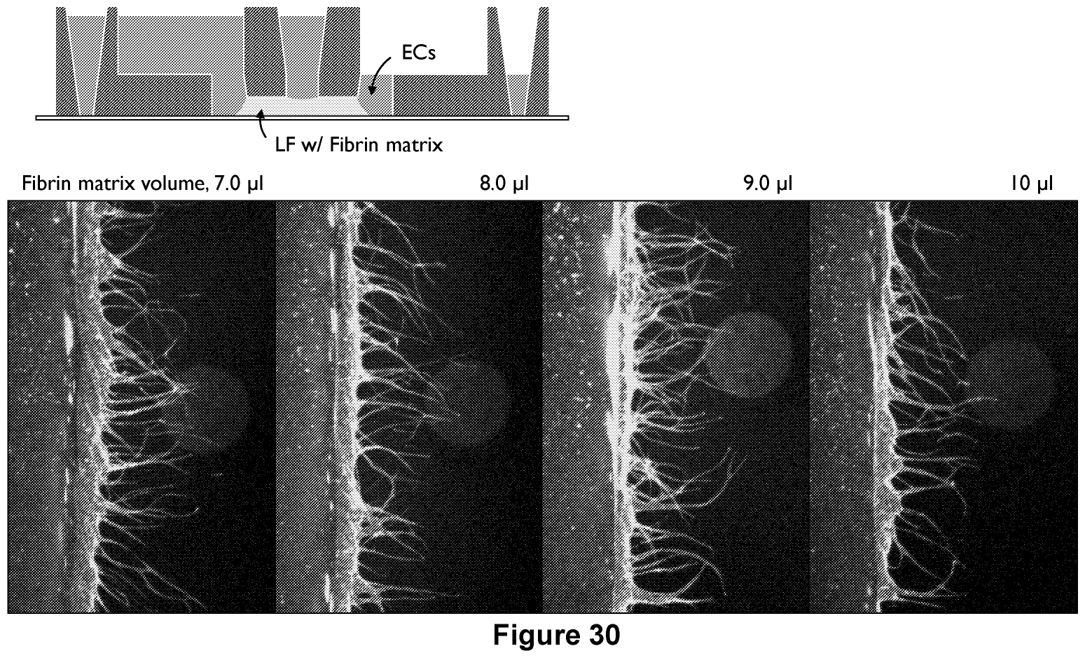

FIG. 30 shows angiogenesis in a tissue grown in a fluidic device in accordance with some embodiments. A mixture of lung fibroblasts and fibrin was provided to the first channel and a solution containing endothelial cells was provided to the second and third channels while the volumes of the solution provided to the second and third channels differed by 100 μL. Even though the volume of the mixture of lung fibroblast and fibrin provided to the first channel was changed (e.g., 7 μL, 8 μL, 9 μL, 10 μL), the volume of the mixture did not affect endothelial cell sprouting.

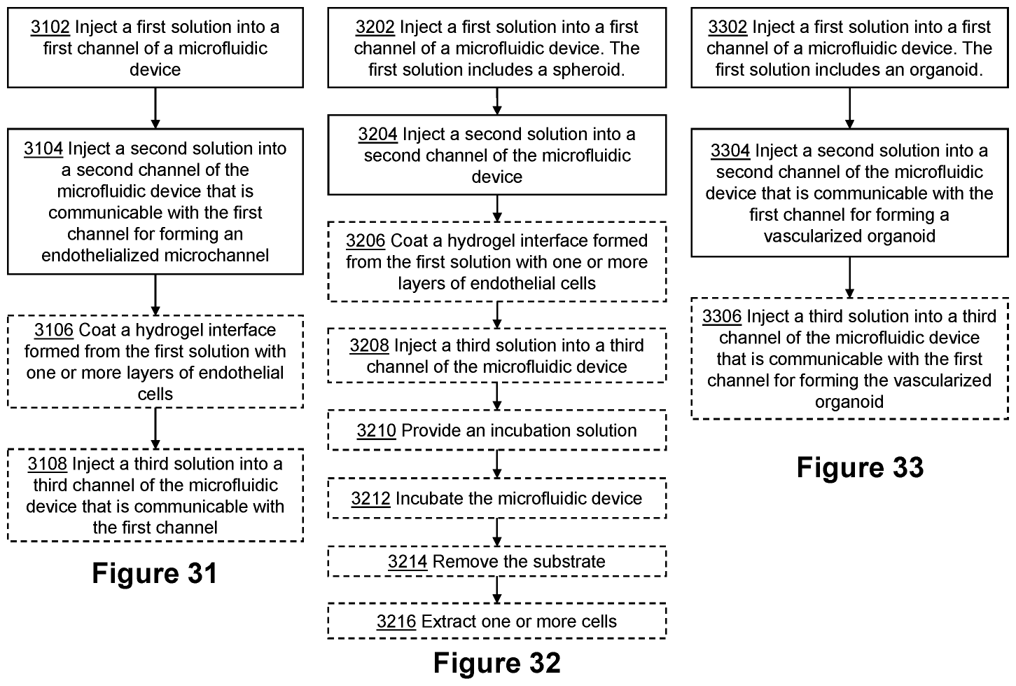

FIG. 31 is a flow diagram illustrating a method 3100 for forming an endothelialized microchannel in accordance with some embodiments.

The method 3100 includes (3102) injecting a first solution into a first channel of a microfluidic device (e.g., a first liquid is injected into the first channel through the injection hole 214 as shown in FIG. 3 or 17B).

The method 3100 also includes (3104) injecting a second solution into a second channel of the microfluidic device that is communicable with the first channel for forming an endothelialized microchannel (e.g., a second liquid is injected into the second channel through the injection hole 216 as shown in FIG. 3 or 17B).

In some embodiments, the microfluidic device is any device described herein (e.g., the device shown in FIG. 1A or 17A).

In some embodiments, the first solution includes hydrogel. In some embodiments, the hydrogel includes one or more of: fibrinogen, collagen, or protein mixture secreted by mouse sarcoma cells (e.g., protein mixture secreted by Engelbreth-Holm-Swarm mouse sarcoma cells, also known as Matrigel).

In some embodiments, the first solution includes endothelial cells (e.g., HUVECs).

In some embodiments, the first solution also includes fibroblast (e.g., lung fibroblasts).

In some embodiments, the first solution includes thrombin. The thrombin causes conversion of fibrinogen to fibrin, which facilitates formation of the vascular networks.

In some embodiments, the second solution includes endothelial cells (e.g., HUVECs).

In some embodiments, the method includes (3106) coating a hydrogel interface formed from the first solution with one or more layers of endothelial cells (e.g., FIG. 22, section (2)). In some embodiments, the method includes coating the hydrogel interface formed from the first solution with only one layer (e.g., a monolayer) of endothelial cells. In some embodiments, the hydrogel interface is coated with the layer of endothelial cells by proliferation of endothelial cells in the second solution.

In some embodiments, the method includes (3108) injecting a third solution into a third channel of the microfluidic device that is communicable with the first channel (e.g., a third liquid is injected into the third channel through the injection hole 218 as shown in FIG. 3 or 17B).

In some embodiments, the third solution includes endothelial cells (e.g., HUVECs).

In some embodiments, the method 3100 includes one or more features described herein with respect to the method 3200 or the method 3300. For example, the method 3100, in some embodiments, includes removing the substrate from the first beam and extracting one or more cells. For brevity, such details are not repeated herein.

FIG. 32 is a flow diagram illustrating a method 3200 for forming a vascularized spheroid in accordance with some embodiments.

The method 3200 includes (3202) injecting a first solution into a first channel of a microfluidic device (e.g., FIG. 9A). The first solution includes a spheroid. In some embodiments, the spheroid is a tumor spheroid.

The method 3200 also includes (3204) injecting a second solution into a second channel of the microfluidic device that is communicable with the first channel for forming a vascularized spheroid.

In some embodiments, the first solution includes hydrogel.

In some embodiments, the hydrogel includes one or more of: fibrinogen, collagen, or protein mixture.

In some embodiments, the first solution includes endothelial cells.

In some embodiments, the first solution includes fibroblast.

In some embodiments, the first solution includes thrombin.

In some embodiments, the second solution includes endothelial cells.

In some embodiments, the method 3200 includes (3206) coating a hydrogel interface formed from the first solution with one or more layers of endothelial cells. In some embodiments, the method 3200 includes coating a hydrogel interface formed from the first solution with a single layer of endothelial cells.

In some embodiments, the method 3200 includes (3208) injecting a third solution into a third channel of the microfluidic device that is communicable with the first channel for forming the vascularized spheroid.

In some embodiments, the third solution includes endothelial cells.

In some embodiments, the method 3200 includes (3210) providing an incubation solution.

In some embodiments, the method 3200 includes (3212) incubating the microfluidic device for forming the vascularized tumor spheroid. In some embodiments, the method 3200 includes the first solution in contact with the second solution. In some embodiments, the method 3200 includes the first solution in contact with the second solution and the third solution.

In some embodiments, the method 3200 includes (3214) removing the substrate from the first beam (e.g., increasing a distance between the first beam and the substrate), and (3216) extracting one or more cells (e.g., one or more cells from the tissue as shown in FIG. 28).

In some embodiments, the method 3200 includes one or more features described herein with respect to the method 3100 or the method 3300. For brevity, such details are not repeated herein.

FIG. 33 is a flow diagram illustrating a method 3300 for forming a vascularized organoid in accordance with some embodiments.

The method 3300 includes (3302) injecting a first solution into a first channel of a microfluidic device, wherein the first solution includes an organoid.

The method 3300 also includes (3304) injecting a second solution into a second channel of the microfluidic device that is communicable with the first channel for forming a vascularized organoid.

In some embodiments, the method 3300 includes (3306) injecting a third solution into a third channel of the microfluidic device that is communicable with the first channel for forming the vascularized organoid.

In some embodiments, the method 3300 includes one or more features described herein with respect to the method 3100 or the method 3200. For example, the method 3300, in some embodiments, includes coating a hydrogel interface formed from the first solution with one or more layers of endothelial cells. For brevity, such details are not repeated herein.

In light of these principles and examples, we turn to certain embodiments.

In accordance with some embodiments, a device includes a substrate (e.g., substrate 106 in FIG. 2D or 17A); a first microfluidic channel defined on the substrate (e.g., first channel 204 in FIG. 2D; first channel 1704 in FIG. 17A); and a second microfluidic channel (e.g., second channel 206 in FIG. 2D; second channel 1706 in FIG. 17A), distinct from the first microfluidic channel, defined on the substrate and in contact with, and substantially parallel to, the first microfluidic channel.

In some embodiments, the substrate has a top surface (e.g., the surface facing the body 102 or the sealer 104) and a bottom surface opposite to the top surface (e.g., the surface facing away from the body 102 or the sealer 104).

In some embodiments, the device includes a first beam (e.g., beam 224 in FIG. 2D; beam 1714 in FIG. 17A) having a bottom surface and side surfaces, the first beam being spaced apart from the top surface of the substrate to define at least the first microfluidic channel.

In some embodiments, the first beam defines a through-hole (e.g., hole 214 in FIG. 2D or 17A) extending between the top surface and the bottom surface of the first beam for receiving a first solution.

In some embodiments, the device includes a second beam (e.g., beam 226 in FIG. 2D; beam 1716 in FIG. 17A) defining the second microfluidic channel.

In some embodiments, the second beam has a top surface and a bottom surface opposite to the top surface, at least a portion of the second beam being spaced apart from the substrate to define the second microfluidic channel between the bottom surface of the second beam and the top surface of the substrate (e.g., beam 226 in FIG. 2D). The second beam is adjacent to the first beam. In some embodiments, the second beam is in contact with the first beam. In some embodiments, the second beam adjoins the first beam along a substantial portion of the length of the third beam.

In some embodiments, the second beam (e.g., beam 1716 in FIG. 17A) has a side surface, the second beam being spaced apart from the first beam to define the second microfluidic channel between a first side surface of the first beam and the side surface of the second beam.

In some embodiments, the second beam is in contact with the substrate (e.g., beam 1716 in FIG. 17A is in direct contact with the substrate 106 or in contact with the substrate 106 through a sealer).

In some embodiments, the second beam is included in a first side structure with a through-hole for receiving a second solution (e.g., beam 1716 in FIG. 17A is included in a side structure 1726 that also has injection hole 216).

In some embodiments, the first beam and the second beam are integrally formed (e.g., the first beam and the second beam are formed concurrently as part of body 102).

In some embodiments, the device includes a third microfluidic channel in contact with and substantially parallel to the first microfluidic channel (e.g., third channel 208 in FIG. 2D or 17A).

In some embodiments, the device includes a third beam defining the third microfluidic channel (e.g., beam 228 in FIG. 2D; beam 1718 in FIG. 17A).

In some embodiments, the third beam has a top surface and a bottom surface opposite to the top surface, at least a portion of the third beam being spaced apart from the substrate to define the third microfluidic channel between the bottom surface of the third beam and the top surface of the substrate (e.g., beam 228 in FIG. 2D). The third beam is adjacent to the first beam. In some embodiments, the third beam is in contact with the first beam. In some embodiments, the third beam adjoins the first beam along a substantial portion of the length of the third beam.

In some embodiments, the third beam (e.g., beam 1718 in FIG. 17A) has a side surface, the third beam being spaced apart from the first beam to define the third microfluidic channel between a second side surface of the first beam and the side surface of the third beam.

In some embodiments, the third beam is in contact with the substrate (e.g., beam 1718 in FIG. 17A is in direct contact with the substrate 106 or in contact with the substrate 106 through a sealer).

In some embodiments, the third beam is included in a second side structure with a through-hole for receiving a third solution (e.g., beam 1718 in FIG. 17A is included in a side structure 1728 that also has injection hole 218).

In some embodiments, the first beam and the third beam are integrally formed (e.g., the first beam and the third beam are formed concurrently as part of body 102).

In some embodiments, the substrate is made of a first material and the first beam is made of a second material, the first material and the second material having surface tensions satisfying a predefined capillary force criterion (e.g., for a certain liquid, such as water).

Some embodiments include the endothelialized microchannel formed by any method described herein.

Some embodiments include the vascularized tumor spheroid formed by any method described herein.

Some embodiments include the vascularized organoid formed by any method described herein.

The microfluidic devices described herein have the following advantages. First, the microfluidic devices can solve problems caused by low gas saturation in three-dimensional cell culture. That is, in the conventional art, since a culture medium in a reservoir is provided to cells in a microfluidic channel through a long and narrow culture medium channel, gas saturation in the culture medium is reduced while the gas provided from the top surface of the culture medium passes through the culture medium channel, and thus an environment disadvantageous for cells is provided. On the other hand, the microfluidic device is connected to facilitate fluid flow with a culture medium through both open sides of a microfluidic channel, and therefore a cell culture environment maintaining high gas saturation may be provided.

In addition, the microfluidic device provides rapid and simple fluid patterning. That is, in the microfluidic device, an inner corner path which facilitates fluid flow by capillary force is formed and connected with the microfluidic channel to facilitate fluid flow, and therefore a suitable amount of the fluid is provided to a selected position on the inner corner path, resulting in easy patterning of the entire microfluidic channels and inner corner paths. Accordingly, the microfluidic device provides a considerably excellent effect on experiment precision, time and utilization. Furthermore, the injection holes facilitate providing solutions to the same positions, and it can be useful when a uniform and reproducible repeated experiment is required. Because the fluid patterning moves until the capillary force applied to the fluid along the inner corner path of the microfluidic device is in equilibrium, when the inner corner path of the microfluidic device has the same contact angle, the fluid patterning can be uniformly performed regardless of external factors such as the experience and skill of an experimenter or operator.

In addition, the microfluidic device may allow patterning of a fluid to a desired area within several seconds, preferably, 1 second after the fluid is applied, and therefore is suitable for an environment requiring rapid and uniform patterning. For example, in three-dimensional co-culture of two or more types of cells, patterning is very important to prevent the mixing of fluids containing different cells. In this case, to fix cells to a specific position under an environment in which free mass transfer is possible, a polymer material, for example, fibrin gel is used together with cells. Here, to cure the fibrin gel mixed with the cells, a generally-used crosslinking agent is added, and for a stable and highly-reliable experiment, rapid and uniform fluid patterning is required.

In addition, the microfluidic device is manufactured of plastic (e.g., an engineering plastic), and the microfluidic device can be manufactured by curing a melted resin by injection molding, hot embossing or 3D printing, and therefore has an advantage of being applicable to economical mass-production. In some embodiments, the plastic is a hydrophilic material. In some embodiments, the plastic is a hydrophobic material.

In some embodiments, the substrate is made of plastic (e.g., an engineering plastic). In some embodiments, the substrate is made of glass.

As described above, the microfluidic device having the above-described structure and advantages does not need other external forces, for example, a pressure, except capillary force, in patterning of a fluid, does not require a separate sensor for precise control of a fluid injection position, and considerably reduces the probability of injection failure, and therefore the microfluidic device can be applied to cell culture using automation equipment.

A microfluidic device, which includes a microfluidic channel embedded in a chamber and open at both sides is manufactured using a material having a hydrophilic surface characteristic, and a fluid can be patterned in a microfluidic channel using capillary force. In accordance with some embodiments, an inner corner path and the microfluidic channel can be used in rapid and precise fluid patterning at one time by applying a suitable amount of the fluid to be patterned on the inner corner path of the microfluidic device. In addition, the microfluidic channel is incorporated or embedded in the lower portion of the chamber, and thus connected to facilitate fluid flow with a culture medium without passing through a long and narrow culture medium channel as shown in the conventional art. Therefore, since cells can easily use a gas entering from an air contact surface, which is on the top surface of the culture medium in the chamber, an advantageous culture environment can be imparted to the cells in the microfluidic channel. Therefore, the microfluidic device can be effectively used in three-dimensional culture of cells or tissue.

This application describes a microfluidic device which includes a microfluidic channel (which is often embedded in a culture medium chamber), and a structure which is formed by capillary force and facilitates fluid flow between an adjacent microfluidic channel and a culture medium. In addition, this application also describes a structure having several microfluidic devices on one common substrate. Furthermore, the microfluidic device may be manufactured of a hydrophobic engineering plastic by injection molding. Accordingly, the microfluidic device may be effectively used in culture of cells, tissue or cells and tissue, required for three-dimensional culture, and therefore, it may be used in general industries such as biotechnology laboratories, cosmetics development and new drug development. This application also describes selected applications of the microfluidic device. It has been demonstrated that the microfluidic device can generate perfusable vascular networks, with or without a spheroid (or an organoid). Some of the examples include vascular networks formed with human brain microvascular endothelial cells and various tumor cells as well as neural networks. Such vascular networks may be used to study various pharmaceutical research, including study of pharmacokinetics. For example, transfer of growth factors, cytokines, and pharmaceuticals in vascular networks can be studied.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the principles and the various described embodiments with various modifications as are suited to the particular use contemplated.

1. A device, comprising:
a substrate having a top surface and a bottom surface opposite to the top surface;
a first microfluidic channel defined on the substrate; and
a second microfluidic channel, distinct from the first microfluidic channel, defined on the substrate and in contact with, and substantially parallel to, the first microfluidic channel.

2. The device of claim 1, including:
a first beam having a bottom surface and side surfaces, the first beam being spaced apart from the top surface of the substrate to define at least the first microfluidic channel.

3. The device of claim 2, wherein:
the first beam defines a through-hole extending between the top surface and the bottom surface of the first beam for receiving a first solution.

4. The device of claim 2, including:
a second beam defining the second microfluidic channel.

5. The device of claim 4, wherein:
the second beam has a top surface and a bottom surface opposite to the top surface, at least a portion of the second beam being spaced apart from the substrate to define the second microfluidic channel between the bottom surface of the second beam and the top surface of the substrate; and
the second beam is adjacent to the first beam.

6. The device of claim 4, wherein:
the second beam has a side surface, the second beam being spaced apart from the first beam to define the second microfluidic channel between a first side surface of the first beam and the side surface of the second beam.

7. The device of claim 6, wherein:
the second beam is in contact with the substrate.

8. The device of claim 4, wherein:
the second beam is included in a first side structure with a through-hole for receiving a second solution.

9. The device of claim 4, wherein:
the first beam and the second beam are integrally formed.

10. The device of claim 4, further comprising:
a third microfluidic channel in contact with and substantially parallel to the first microfluidic channel.

11. The device of claim 10, including:
a third beam defining the third microfluidic channel.

12. The device of claim 11, wherein:
the third beam has a top surface and a bottom surface opposite to the top surface, at least a portion of the third beam being spaced apart from the substrate to define the third microfluidic channel between the bottom surface of the third beam and the top surface of the substrate; and
the third beam is adjacent to the first beam.

13. The device of claim 11, wherein:
the third beam has a side surface, the third beam being spaced apart from the first beam to define the third microfluidic channel between a second side surface of the first beam and the side surface of the third beam.

14. The device of claim 13, wherein:
the third beam is in contact with the substrate.

15. The device of claim 11, wherein:
the third beam is included in a second side structure with a through-hole for receiving a third solution.

16. The device of claim 11, wherein:
the first beam and the third beam are integrally formed.

17. The device of claim 2, wherein:
the substrate is made of a first material and the first beam is made of a second material, the first material and the second material having surface tensions satisfying a predefined capillary force criterion.

18. A method for forming an endothelialized microchannel, the method comprising:
injecting a first solution into a first channel of the device of claim 1; and
injecting a second solution into a second channel of the device that is communicable with the first channel for forming an endothelialized microchannel.

19. (canceled)

20. (canceled)

21. (canceled)

22. (canceled)

23. (canceled)

24. (Canceled)

25. (canceled)

26. (Canceled)

27. A method for forming a vascularized spheroid, the method comprising:
injecting a first solution into a first channel of the device of claim 1, wherein the first solution includes a spheroid; and
injecting a second solution into a second channel of the device that is communicable with the first channel for forming a vascularized spheroid.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method for forming a vascularized organoid, the method comprising:
injecting a first solution into a first channel of the device of claim 1, wherein the first solution includes an organoid; and
injecting a second solution into a second channel of the device that is communicable with the first channel for forming a vascularized organoid.

42. (canceled)

43. (canceled)

44. (canceled)

45. (canceled)
